(19) United States
(12) Patent Application Publication
(10) Pub. No.: US 2003/0208322 A1
(43) Pub. Date: Nov. 6, 2003

(54) Apparatus, method, and computer program product for plotting proteomic and genomic data

(76) Inventors: Kiyoko F. Aoki, Playa Del Rey, CA (US); Bruce Hoff, Venice, CA (US); Soheil Shams, Redondo Beach, CA (US)

Correspondence Address: TOPE-MCKAY & ASSOCIATES, 23852 PACIFIC COAST HIGHWAY #311, MALIBU, CA, 90265, US

(21) Appl. No.: 10/040,210

(22) Filed: Jan. 2, 2002

(51) Int. Cl. G06G 7/48; G06G 7/58; G06F 19/00; G01N 33/48; G01N 33/50

(52) U.S. Cl. 702/19; 702/20

(57) ABSTRACT

An apparatus, a method, and a computer program product for plotting proteomic and genomic data are presented. The apparatus comprises a computer system for generating data to display in a visual form. The computer system receives a set of proteomic and genomic data including data samples and schemes for partitioning the data samples into data partitions. Various operations are performed by the computer system in response to user commands, including adjusting the view of partition schemes in response to the selection of a particular partition scheme in order to allow a user to visually detect correlations among data. The system also allows for the performance of set operations on the proteomic and genomic data, and for displaying the results. Additionally, the computer system allows for operations for determining partition schemes and partitions in which a particular data sample are located, and for generating and modifying partition schemes.

### BACKGROUND

(1) Technical Field

The present invention relates to the field of bio-informatics, and more particularly to a visual tool for the analysis of proteomic and genomic information.

(2) Discussion

The bioinformatics field, which, in a broad sense, includes any use of computers in solving information problems in the life sciences, and more particularly, the creation and use of extensive electronic databases on genomes, proteomes, etc., is currently in a stage of rapid growth. Many of the mathematical and statistical techniques applied in analyzing life science information problems are foreign to the life scientists using them. Thus, it is desirable to provide mechanisms which these users can operate and from which they can draw intuitive conclusions regarding data.

In the area of statistical analysis of proteomic and genomic information (such as information derived through the use of microarrays), this problem is particularly acute. While statistical software packages are available for analyzing the data, often their output is cryptic to their users. For example, software packages currently on the market facilitate the partitioning of data based on certain classifications by creating gene lists. However, there is no clear graphical way to view the relationship between the various gene lists in order to visually detect correlations among the data.

The main objective of a life scientist using these packages is to find “interesting genes” from an experiment. For example, given a partitioning of a group of people according to the sex, a life scientist may desire to compare how the same individuals are partitioned based on another criterion, such as age. Unfortunately, current techniques provide no simple solution to this need, and the processes used are neither simple nor intuitive.

Therefore, it would be desirable to provide a technique for presenting proteomic and genomic information in a way that would allow a user to intuitively determine relationships among data when viewed from different partitioning schemes. Furthermore, it would be useful to provide an easy and visual means for allowing set operations on data in the partitioning schemes. It is an object of the present invention to provide such a technique.

### SUMMARY OF THE INVENTION

The present invention relates to the field of bio-informatics, and more particularly to a visual tool for the analysis of proteomic and genomic information. In this regard, the present invention provides an apparatus, a method, and a computer program product for plotting proteomic and genomic data.

The apparatus comprises a computer system including a processor, a memory coupled with the processor, an input coupled with the processor for receiving proteomic and genomic data and for receiving user input, and an output coupled with the processor for displaying data in a visual form. Resident in the memory and processor of the computer system are means for: receiving a set of proteomic and genomic data including data samples with characteristics; receiving at least one partition scheme, with each partition scheme including at least one partition into which a portion of the data is to be grouped based on a characteristic; generating a graphical representation of the relative size of each partition in each partition scheme; accepting a user selection of a particular partition scheme; and, in response, adjusting the view of all other partition schemes to reflect the distribution of the characteristic used as the basis of the selected partition scheme; and outputting a graphical representation of the results. This allows a user to view a graphical representation of the distribution of a particular characteristic based on a particular partitioning scheme, while simultaneously viewing a graphical representation of the distributions of other characteristics based on other partitioning schemes, and to adjust the views of the other characteristics based on the particular characteristic in order to visually detect correlations between characteristics.

In a preferred aspect, the present invention may be used to generate the graphical representation in the form of pie-type charts, each with pie slices, with each pie slice representing the portion of the overall pie attributable to a particular partition of a partition scheme.

In a more preferred aspect, the means for generating a graphical representation generates a graphical representation in the form of a plurality of pie chart groups, with each pie chart group including a partition scheme pie chart representing the overall distribution a characteristic selected, and a plurality of partition pie charts, each representing a partition within the partition scheme pie chart.

In a still more preferred aspect, the means for accepting a user selection of a particular partition scheme accepts a user selection of a particular partition scheme by the user graphically selecting a pie chart group among the plurality of pie chart groups. Also, the means for adjusting the view of all other partition schemes to reflect the distribution of the characteristic used as the basis of the selected partition scheme adjusts all of the pie chart groups such that the partition scheme pie charts and the partition pie charts are all partitioned according to the partition scheme of the selected pie chart group. Thus, the pie charts may be compared visually to determine possible correlations therebetween.

The present invention may also include a union function in which the computer system further includes means for: receiving a user selection of multiple partition pie charts; receiving a user request to unite the partition pie charts into a united group consisting of the data present in all of the multiple partition pie charts selected by the user; and generating a list of the data in the united group. As a result of the union operation, the present invention may also include means for generating and displaying a pie chart based on the data in the united group and partitioned into two slices, with one slice representing the number of data samples in the united group and the other slice representing the number of data samples in the set of proteomic and genomic data minus the number of data samples in the united group. Thus, a user may view the number of samples in the united group and its complement in a single pie chart. The computer system may further include means for generating a new partition scheme with one partition representing the number of data samples in the united group and the other partition representing the number of data samples in the set of proteomic and genomic data minus the number of data samples in the united group.

In addition to the union operation, the computer system of the present invention may also comprise means for: receiving a user selection of multiple partition pie charts; receiving a user request to intersect the partition pie charts into an intersected group consisting of data present in all of the multiple partition pie charts selected by the user; and generating a list of the data in the intersected group. As a result of the intersection operation, the present invention may also include means for generating and displaying a pie chart based on the data in the intersected group and partitioned into two slices, with one slice representing the number of data samples in the united group and the other slice representing the number of data samples in the set of proteomic and genomic data minus the number of data samples in the intersected group. Thus, a user may view the number of samples in the intersected group and its complement in a single pie chart. Furthermore, the present invention may also include means for generating a new partition scheme with one partition representing the number of data samples in the intersected group and the other partition representing the number of data samples in the set of proteomic and genomic data minus the number of data samples in the intersected group.

In still another aspect, the present invention provides means for displaying a list of data used for the generation of a particular partition pie chart when the partition pie chart is selected, thus allowing a user to view a partition pie chart and also to examine the underlying data.

In yet another aspect, the present invention provides means for receiving a user request to search for a particular piece of data and, in response to the user request, for indicating the pie charts in which the particular piece of data is present. Thus, a user may visually determine which pie chart groups include the particular piece of data.

The above discussion of the aspects of the present invention is centered on the use of pie charts because they are preferred. However, it is important to note that any other type of chart (e.g. bar charts) may be used.

The present invention may also be embodied as a method, in which the “means” discussed above are interpreted as steps operated on a data processing (computer) system or as a computer program product, in which the “means” discussed above are recorded on a computer readable medium such as an optical storage device (e.g., a CD or DVD).

The “means” of the present invention are generally in the form of program logic that may be embodied as computer program code or may be embedded in hardware depending on the needs of a particular embodiment.

### BRIEF DESCRIPTION OF THE DRAWINGS

These and other features, aspects, and advantages of the present invention will become better understood with regard to the following description, appended claims, and accompanying drawings where:

FIG. 1 is a block diagram depicting the components of a computer system used in the present invention;

FIG. 2 is an illustrative diagram of a computer program product embodiment of the present invention;

FIG. 3 is a flow chart depicting the steps (means) of the present invention;

FIG. 4 is a screenshot depicting a graphical user interface displaying output from the present invention prior to any user operations (or after a reset operation); and

FIG. 5 is a screenshot of a graphical user interface displaying output from the present invention after an operation of generating a display output adjusting the view of all partition schemes other than the currently selected one according to the scheme of the selected one 318.

### DETAILED DESCRIPTION

The present invention relates to the field of bio-informatics, and more particularly to a visual tool for the analysis of proteomic and genomic information. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In order to provide a working frame of reference, first a glossary of some of the terms used in the description and claims is given as a central resource for the reader. The glossary is intended to provide the reader with a “feel” for various terms as they are used in this disclosure, but is not intended to limit the scope of these terms. Rather, the scope of the terms is intended to be construed with reference to this disclosure as a whole and with respect to the claims below. Then, a brief introduction is provided in the form of a narrative description of the present invention to give a conceptual understanding prior to developing the specific details.

(1) Glossary

Before describing the specific details of the present invention, it is useful to provide a centralized location for various terms used herein and in the claims. The terms defined are as follows:

Chart—The term “chart” as used with respect to this invention generally indicates a diagram that exhibits a relationship, often functional, between two pieces of data, such as a set of points having coordinates determined by the relationship. This type of chart is commonly called a “plot”. Preferably, a “chart” is in the form of a pictoral device, examples of which include pie charts or graphs, used to illustrate quantitative relationships. Combinations of different types of charts may also be used. The preferred type of chart for purposes of the present invention is the pie chart.

Means—The term “means” as used with respect to this invention generally indicates a set of operations to be performed on a computer. Non-limiting examples of “means” include computer program code (source or object code) and “hard-coded” electronics. The “means” may be stored in the memory of a computer or on a computer readable medium.

(2) Introduction

Data analyzed by microarray experiments are often grouped so that similar data are clustered together. The relationships between these groups are difficult to visualize merely from an inspection of the underlying data. The present invention overcomes this difficulty by providing a system that allows for several partitions (groups) to be compared with one another to give the user a better understanding of the relationships between the different partitions. In the present invention, proteomic and genomic data is grouped into partitions, with a group of related partitions forming a partition scheme. For example, a partition scheme may be used to group proteomic data by the age of an individual from which the data was obtained. Various partitions could be used within the partition scheme to classify individual data samples. For example, in this case, partitions may include data samples from individuals aged 0 to 25, 26 to 50, and 50+. Thus, in this “age” partition scheme, there are three partitions. Partition schemes may be created for use by the present invention in many different ways, non-limiting examples of which include use of statistical clustering tools or manual data manipulation.

The present invention provides a mechanism that allows a user to easily and intuitively determine correlations among characteristics of proteomic and genomic information. In a preferred embodiment, the various partitions in a partition scheme are depicted as slices in a pie chart. Several partition schemes may be viewed simultaneously in order to allow for a quick visual comparison in order to detect correlations among characteristics in the data that form the basis of the partition schemes. The data may be partitioned into partition schemes, for example, based on particular characteristics of individuals from which the data was obtained or it may be partitioned arbitrarily. In addition to displaying partition schemes in the form of pie charts, the preferred embodiment also allows for set operations on the data included in partitions, such as union and intersection functions. Also, a list of the data in a particular partition may be displayed by selecting the partition.

(3) Physical Embodiments of the Present Invention

The present invention has three principal “physical” embodiments. The first is an apparatus for plotting proteomic and genomic information, typically in the form of a computer system operating software of in the form of a “hard-coded” instruction set. The second physical embodiment is a method, typically in the form of software, operated using a data processing system (computer). The third principal physical embodiment is a computer program product. The computer program product generally represents computer readable code stored on a computer readable medium such as an optical storage device, e.g., a compact disc (CD) or digital versatile disc (DVD), or a magnetic storage device such as a floppy disk or magnetic tape. Other, non-limiting examples of computer readable media include hard disks and flash-type memories. These embodiments will be described in more detail below.

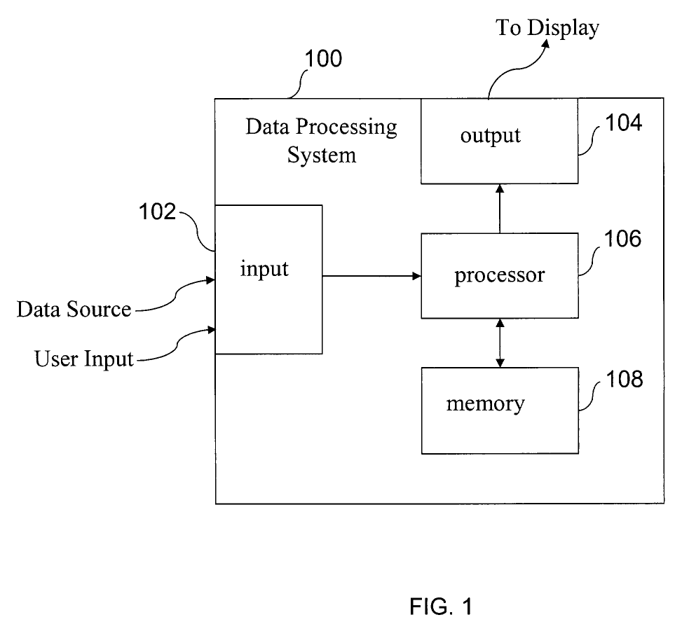

A block diagram depicting the components of a computer system used in the present invention is provided in FIG. 1. The data processing system 100 comprises an input 102 for receiving proteomic and genomic data from a data source and for receiving user input from an input device such as a keyboard. Note that the input 102 may include multiple “ports” for receiving data and user input. Typically, user input is received from traditional input/output devices such as a mouse, trackball, keyboard, light pen, etc., but may also be received from other means such as voice or gesture recognition for example. The output 104 is connected with the processor for providing output. Output to a user is preferably provided on a video display such as a computer screen, but may also be provided via printers or other means. Output may also be provided to other devices or other programs for use therein. The input 102 and the output 104 are both coupled with a processor 106, which may be a general-purpose computer processor or a specialized processor designed specifically for use with the present invention. The processor 106 is coupled with a memory 108 to permit storage of data and software to be manipulated by commands to the processor.

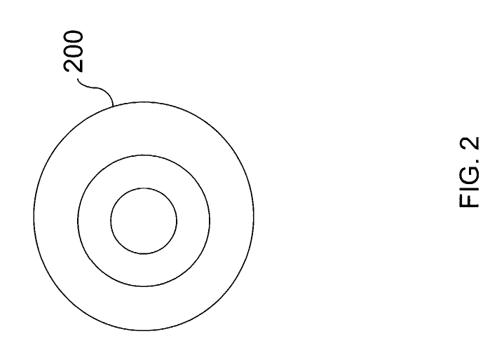

An illustrative diagram of a computer program product embodying the present invention is depicted in FIG. 2. The computer program product 200 is depicted as an optical disk such as a CD or DVD. However, as mentioned previously, the computer program product generally represents computer readable code stored on any compatible computer readable medium.

(4) The Preferred Embodiments

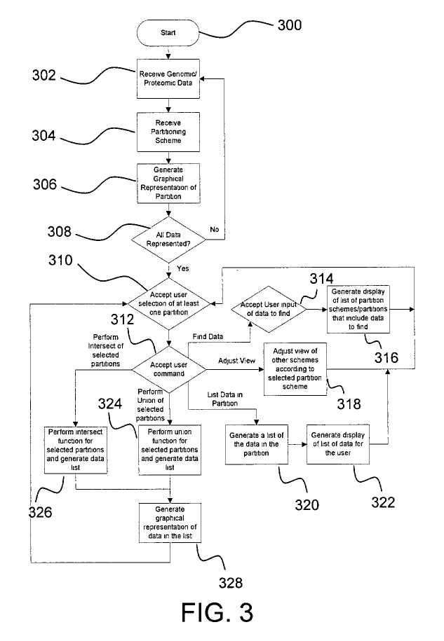

As stated previously, the present invention provides an apparatus, a method, and a computer program product for visually analyzing genomic and proteomic data. A flow chart depicting the steps of the present invention is depicted in FIG. 3. Note that the steps of the flow chart map directly to the “means” in the apparatus and the computer program product embodiments.

A start 300 is provided as a point of reference for the flow chart. Note that although the flow diagram is presented with a particular order, many variations of the order will be readily apparent to one of skill in the art without departing from the spirit of the present invention. Therefore, the ordering of the flow chart is presented simply as an example embodiment including the steps of the preferred embodiment.

After the start 300, a step of receiving genomic and/or proteomic data 302 is performed in which the data is received in the memory 108 of the computer system 100 through the input 102 and the processor 106. Typically, the data is received from a database source, which may be resident in the memory 108 or may be remotely located therefrom. A step of receiving a partitioning scheme 304 is performed in which at least one partition scheme for the data is received. The partition scheme comprises partitions into which data is divided. Typically, the partition scheme represents variations of a particular characteristic of the data, with the individual discrete variations indicated by partitions. Thus, the partitions may be likened to bins into which data are sorted by some sorting scheme. A partition scheme may include the entire data set or a subset of the data. Typically, partition schemes are imported into the present invention; however, utilities may also be integrated with the present invention to allow for the creation of partition schemes as will be discussed below. After the partitioning schemes have been received, a step of generating a graphical representation of the partition schemes and their partitions 306 is performed in which a chart of each partition scheme is developed and outputted for display in a display device (typically, a computer screen). These steps continue until all of the data and all of the partition schemes have been represented. In the flow chart, a decision block for checking whether all data has been represented 308 is provided for this purpose.

After all of the data has been represented 308, the computer system 100 awaits user input through the input 102. User input is typically provided via a standard computer input such as a mouse and keyboard combination. In order to begin performing the functions available through the present invention, the computer system 100 accepts a user selection of at least one partition or partition scheme 310 and a user command 312 to perform an operation on the partition or the partition scheme.

Operations available to a user include finding partitions and partition schemes including a particular piece of data; adjusting the views of partition schemes other than one selected according to the selected partition scheme; listing the data in a selected partition or partition scheme; performing a union function on selected partitions; and performing an intersection function on selected partitions. Most of these functions are performed in response to a user selection of one or more partition schemes and a user command. After performance of a function, the computer system 100 awaits another user selection and command. The user may also start the process over by importing another data set or by supplementing the current data set.

When a find data command is provided by a user, the user inputs or otherwise indicates a piece of data to find 314. The computer system 100, in response, generates a list of partition schemes and partitions that include the data sought for display on a display device 316. The list generated may be in the form of a textual list providing identifying information regarding the partition schemes and partitions in which the data is present. On the other hand, the “list” may also include a graphical indication of the partition schemes or partitions, for example by highlighting the associated portions of the charts representing the partition schemes or partitions in which the data is present. The exact method used for indicating the partition schemes and partitions in which the data is present may be tailored to the needs of a particular embodiment.

When a command for adjusting the view of the partition schemes according to a selected partition scheme is issued by a user, the computer system 100 performs a step of generating a display output in which the views of all partition schemes, other than the currently selected one, are adjusted according to the scheme of the selected one 318. Preferably, charts are provided in clusters, with one chart representing the partition scheme and other charts representing the partition scheme and the content of the individual partitions in the partition scheme. In this case, when the adjustment is performed, the charts representing the content of the individual partitions in partition schemes other than the one selected are adjusted to reflect the portions attributable to the data included in the partitions of the partition scheme selected. This operation allows a user to visually compare the contents of different partition schemes and partitions in order to detect correlations among different characteristics of the data. This feature will be illustrated more clearly with a graphical example in the next subsection.

When a command for listing data in a selected partition scheme or partition is issued by a user, the computer system 100, in response, performs a step of generating a list of the data that forms the basis for (is included in) the selected partition scheme or partition 320. Display data is then generated and outputted in a step of generating a display of the list of data for the user 322.

Two other operations include a step of performing a union function on selected partition schemes and generating a data list therefrom 324 and a step of performing an intersection function on selected partition schemes and generating a data list therefrom 326. The union and intersection functions are set operations. In cases where the charts used are Venn diagrams, the combinations and overlaps (unions and intersections) of partition schemes may be visually represented in addition to charts representing the final results of the operations. Once a union or intersection function operation has been performed, a step of generating a graphical representation of the result of the union or intersection operation for output to a display device 328 is performed. The result of a union or intersection operation is a preferably in the form of a list of the data in the united or intersected set and a list of its complement either with respect to the data in the partition schemes that were united or intersected or with respect to the whole data set. The graphical representation of the result of a union or the intersection operation is a chart or graph of a partition scheme having two partitions—one representing the set of data representing the united or intersected data and the other representing its complement. The result of a union or intersect operation may also be used as the basis for a new partition scheme, which may be used like any other partition scheme received in the step of receiving at least one partition scheme 304.

As stated before, after each operation, the computer system 100 resumes waiting to accept a user selection of at least one partition 310 or to accept another user command 312. Note that although it is preferred that an embodiment of the present invention include the ability to perform all of the operations mentioned above, it is possible that only a subset be included. Furthermore, it is desirable to allow a user to “prune” the data set by eliminating desired data from inclusion in desired partition schemes (or possibly from the entire data set).

Finally, the charts generated by the present invention are preferably pie charts where a partition scheme pie chart represents each partition scheme, with slices of the pie chart representing (typically via color codes or patterns) the partitions within the partition scheme. Additional pie charts are also provided as partition pie charts, which represent the data contents of each partition in the partition scheme (color coded or patterned in correspondence with the slices of the associated partition scheme pie chart). The partition scheme pie charts and the partition pie charts are typically presented in a graphical user interface as two-level trees, where the top level displays the partition scheme pie charts and the children are the partition pie charts that each represent the contents of a slice (partition) of their parent partition scheme pie chart. Other charts could be used as well; for example, a stacked bar chart could be used with bars representing partition schemes and slices or portions of the bar representing the partitions within the partition scheme.

Next, an illustrative example will be provided to demonstrate the display output of an application of the present invention as incorporated into a graphical user interface of a computer program operating on a computer system 100.

(5) An Illustrative Example

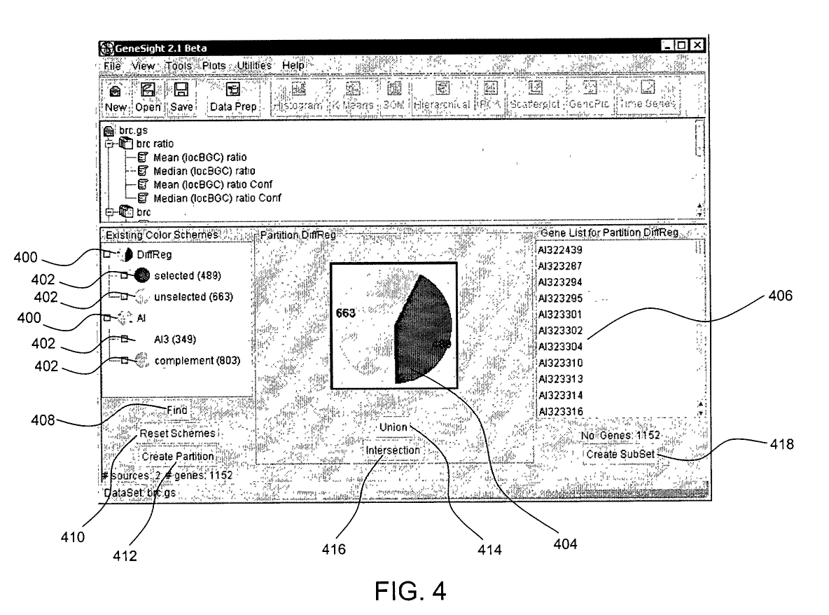

A screen shot depicting a graphical user interface displaying output from the present invention is presented in FIG. 4. This figure depicts the output of the computer system 100 of the present invention prior to any operations. Two partition scheme pie charts 400 are depicted, along with corresponding partition pie charts 402. The pie charts are displayed in tree-like structures, with the partition pie charts 402 subordinate to the partition scheme pie charts 400. Each of the partition schemes in the example includes two partitions. The partition pie charts 402 are each color-coded to represent the data contents of a slice (partition) of the corresponding partition scheme pie chart 400. In the center of the output screen, an enlarged view of the selected partition scheme 404 is presented. Also, a list of the data in a selected partition or partition scheme 406 is displayed. Finally, buttons for performing various functions are provided, including a button for initiating a find operation 408; a button for resetting the partition schemes to their original state 410; a button for facilitating the creation of a partition scheme 412; a button for performing a union operation 414; a button for performing an intersection operation 416; and a button for creating a subset of the data in a particular partition scheme or partition 418.

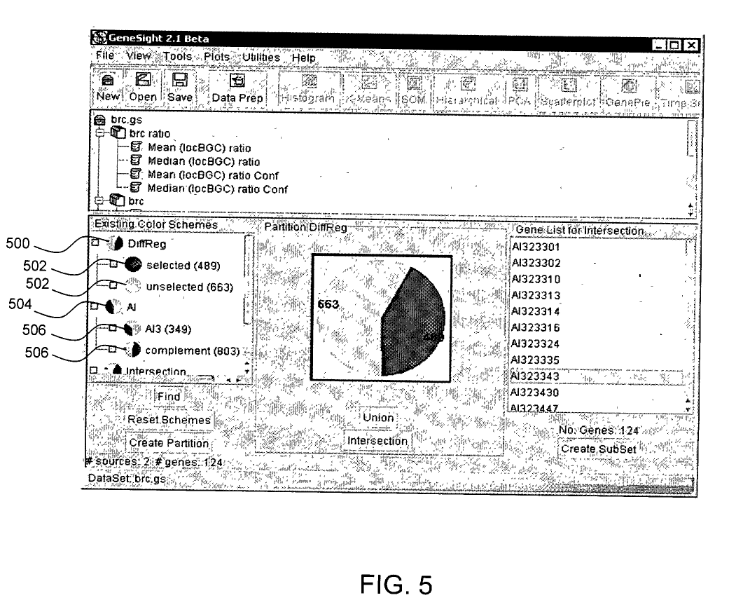

FIG. 5 provides a screenshot of a graphical user interface displaying output from the present invention, after an operation of generating a display output adjusting the view of all partition schemes other than the currently selected one according to the scheme of the selected one 318. The color scheme of the partition scheme pie chart 500 used as the basis for the adjustment and its related partition pie charts 502 are used as the color scheme for all other pie chart groups. The other partition scheme pie chart 504 and the corresponding partition pie charts 506 depicted in the figure are color coded to indicate the relative portions of the data in the partition pie charts 502 of the selected partition pie chart 500 included therein. The result of this adjustment may be made more apparent by comparing the partition scheme pie charts 400 and the partition pie charts 402 with the adjusted partition scheme pie chart 504 and partition pie charts 506. Note that the selected partition scheme pie chart 500 and its respective partition pie charts 502 are unchanged with respect to FIG. 4, as they are used as the basis for change of all other pie charts. Thus, various characteristics of the data may be compared to determine if there is correlation between different partition schemes.

Referring again to FIG. 4, button for initiating a find operation 408 allows a user to find partition schemes and partitions containing a particular data sample. A user may simply select a data sample from list of the data in a selected partition or partition scheme 406 and then “click” on the button to perform the operation. The result may be in the form of a list of the partition schemes and partitions that include the data, or it may be in the form of highlighting or color-coding the partition schemes and partitions that include the data.

The button for resetting the partition schemes to their original state 410 can be used after an operation of generating a display output adjusting the view of all partition schemes other than the currently selected one according to the scheme of the selected one 318 has been performed in order to reset to the state depicted in FIG. 4.

The button for facilitating the creation of a partition scheme 412 can be used to generate a new partition scheme from a list of data and its complement with respect to the entire data set. As an example, a user could display a list of the data in a selected partition or partition scheme 406 (or in the whole data set), and could then delete data samples from, or add data samples to the list. Once the desired list is compiled, the user could simply click on the button for facilitating the creation of a partition scheme 412, and the partition scheme would be created and added to the list of pie charts with a new partition scheme pie chart and corresponding partition pie charts. It is possible that partition schemes could then be subdivided into other partitions so that the user could generate an entirely new partition scheme with which to group the data. This is possible by selecting the desired data and clicking on the button for creating a subset of the data in a particular partition scheme or partition 418.

In some embodiments, it may be undesirable to allow a user to directly modify the list of data. In this case, in order to create a partition scheme 412, a user need only select a group of pie charts in the pie chart tree and click on the button for facilitating the creation of a pie chart scheme 412. The data included in each selected pie chart becomes a partition in the new partition scheme. In some cases, data duplicates may occur in the list of data due to duplicates within the group of selected pie charts. The case of data duplicates may be handled in a number of different ways, examples of which include allowing the duplicates to co-exist, keeping only the first occurrence of a duplicate, or eliminating all members of the duplicates from the list of data.

The button for performing a union operation 414 and the button for performing an intersection operation 416 operate similarly. In the example presented in FIGS. 4 and 5, a user would simply select the desired partitions to be united or intersected, and then would click the appropriate button. A new partition scheme including either the united data or the intersected data, and its complement with respect to the entire data set is generated and preferably shown in the enlarged view portion 404 of the display. Additionally, the result of the union or intersection operation is preferably displayed as a list of the data in a selected partition or partition scheme 406.

This example of the present invention is provided for the purpose of presenting the invention in its best mode. It should be apparent to one of skill in the art that various modifications may be made to tailor the present invention for use in particular applications. Additional features may be added or fewer features may be provided without departing from the spirit of the present invention. The claimed inventive aspects of the present invention are set forth in the claims below.

What is claimed is:

1. An apparatus for plotting proteomic and genomic data, the apparatus comprising a computer system including a processor, a memory coupled with the processor, an input coupled with the processor for receiving proteomic and genomic data and for receiving user input, and an output coupled with the processor for displaying data in a visual form, wherein the computer system further comprises means, residing in its processor and memory, for:
a. receiving a set of proteomic and genomic data including data samples with characteristics;
b. receiving at least one partitioning scheme, with each partition scheme including at least one partition into which a portion of the data is to be grouped based on a characteristic;
c. generating a graphical representation of the relative size of each partition in each partition scheme;
d. accepting a user selection of a particular partition scheme; and, in response,
e. adjusting the view of all other partition schemes to reflect the distribution of the characteristic used as the basis of the selected partition scheme; and
f. outputting a graphical representation; whereby a user can view a graphical representation of the distribution of a particular characteristic based on a particular partitioning scheme.

2. An apparatus for plotting proteomic and genomic data as set forth in claim 1, wherein the means for generating a graphical representation generates a graphical representation in the form of pie-type charts, each with pie slices, with each pie slice representing the portion of the overall pie attributable to a particular partition of a partition scheme.

3. An apparatus for plotting proteomic and genomic data as set forth in claim 1, wherein the means for generating a graphical representation generates a graphical representation in the form of a plurality of pie chart groups, with each pie chart group including a partition scheme pie chart representing the overall distribution a characteristic selected, and a plurality of partition pie charts, each representing a partition within the partition scheme pie chart.

4. An apparatus for plotting proteomic and genomic data as set forth in claim 3, wherein the means for accepting a user selection of a particular partition scheme accepts a user selection of a particular partition scheme by the user graphically selecting a pie chart group among the plurality of pie chart groups, and wherein the means for adjusting the view of all other partition schemes to reflect the distribution of the characteristic used as the basis of the selected partition scheme adjusts all of the pie chart groups such that the partition scheme pie charts and the partition pie charts are all partitioned according to the partition scheme of the selected pie chart group; whereby the pie charts may be compared visually to determine possible correlations therebetween.

5. An apparatus for plotting proteomic and genomic data as set forth in claim 4, further comprising means for:
a. receiving a user selection of multiple partition pie charts;
b. receiving a user request to unite the partition pie charts into a united group consisting of the data present in all of the multiple partition pie charts selected by the user; and
c. generating a list of the data in the united group.

6. An apparatus for plotting proteomic and genomic data as set forth in claim 5, further including means for generating and displaying a pie chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single pie chart.

7. An apparatus for plotting proteomic and genomic data as set forth in claim 6, further including means for generating a new partition scheme with one partition representing the number of data samples in the united group and the other partition representing the number of data samples in the set of proteomic and genomic data minus the number of data samples in the united group.

8. An apparatus for plotting proteomic and genomic data as set forth in claim 4, further comprising means for:
a. receiving a user selection of multiple partition pie charts;
b. receiving a user request to intersect the partition pie charts into an intersected group consisting of data present in all of the multiple partition pie charts selected by the user; and
c. generating a list of the data in the intersected group.

9. An apparatus for plotting proteomic and genomic data as set forth in claim 8, further including means for generating and displaying a pie chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single pie chart.

10. An apparatus for plotting proteomic and genomic data as set forth in claim 9, further including means for generating a new partition scheme with one partition representing the number of data samples in the intersected group and the other partition representing the number of data samples in the set of proteomic and genomic data minus the number of data samples in the intersected group.

11. An apparatus for plotting proteomic and genomic data as set forth in claim 4, further including means for displaying a list of data used for the generation of a particular partition pie chart when the partition pie chart is selected; whereby a user may view a partition pie chart and may also examine the underlying data.

12. An apparatus for plotting proteomic and genomic data as set forth in claim 1, further including means for receiving a user request to search for a particular piece of data and, in response to the user request, for indicating the pie charts in which the particular piece of data is present; whereby a user may visually determine which pie chart groups include the particular piece of data.

13. An apparatus for plotting proteomic and genomic data as set forth in claim 4, further comprising means for:
a. receiving a user selection of multiple partition pie charts;
b. receiving a user request to unite the partition pie charts into a united group consisting of the data present in all of the multiple partition pie charts selected by the user; and
c. generating a list of the data in the united group.

14. An apparatus for plotting proteomic and genomic data as set forth in claim 13, further including means for generating and displaying a pie chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single pie chart.

15. An apparatus for plotting proteomic and genomic data as set forth in claim 14, further including means for generating a new partition scheme with one partition representing the number of data samples in the united group and the other partition representing the number of data samples in the set of proteomic and genomic data minus the number of data samples in the united group.

16. An apparatus for plotting proteomic and genomic data as set forth in claim 15, further comprising means for:
a. receiving a user selection of multiple partition pie charts;
b. receiving a user request to intersect the partition pie charts into an intersected group consisting of data present in all of the multiple partition pie charts selected by the user; and
c. generating a list of the data in the intersected group.

17. An apparatus for plotting proteomic and genomic data as set forth in claim 16, further including means for generating and displaying a pie chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single pie chart.

18. An apparatus for plotting proteomic and genomic data as set forth in claim 17, further including means for generating a new partition scheme with one partition representing the number of data samples in the intersected group and the other partition representing the number of data samples in the set of proteomic and genomic data minus the number of data samples in the intersected group.

19. An apparatus for plotting proteomic and genomic data as set forth in claim 18, further including means for displaying a list of data used for the generation of a particular partition pie chart when the partition pie chart is selected; whereby a user may view a partition pie chart and may also examine the underlying data.

20. An apparatus for plotting proteomic and genomic data as set forth in claim 19, further including means for receiving a user request to search for a particular piece of data and, in response to the user request, for indicating the pie charts in which the particular piece of data is present; whereby a user may visually determine which pie chart groups include the particular piece of data.

21. An apparatus for plotting proteomic and genomic data as set forth in claim 1, wherein the means for generating a graphical representation generates a graphical representation in the form of a graphical chart, each with chart slices, with each chart slice representing the portion of the overall chart attributable to a particular partition of a partition scheme.

22. An apparatus for plotting proteomic and genomic data as set forth in claim 1, wherein the means for generating a graphical representation generates a graphical representation in the form of a plurality of chart groups, with each chart group including a partition scheme chart representing the overall distribution a characteristic selected, and a plurality of partition charts, each representing a partition within the partition scheme chart.

23. An apparatus for plotting proteomic and genomic data as set forth in claim 22, wherein the means for accepting a user selection of a particular partition scheme accepts a user selection of a particular partition scheme by the user graphically selecting a chart group among the plurality of chart groups, and wherein the means for adjusting the view of all other partition schemes to reflect the distribution of the characteristic used as the basis of the selected partition scheme adjusts all of the chart groups such that the partition scheme charts and the partition charts are all partitioned according to the partition scheme of the selected chart group; whereby the charts may be compared visually to determine possible correlations therebetween.

24. An apparatus for plotting proteomic and genomic data as set forth in claim 23, further comprising means for:
a. receiving a user selection of multiple partition charts;
b. receiving a user request to unite the partition charts into a united group consisting of the data present in all of the multiple partition charts selected by the user; and
c. generating a list of the data in the united group.

25. An apparatus for plotting proteomic and genomic data as set forth in claim 24, further including means for generating and displaying a chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single chart.

26. An apparatus for plotting proteomic and genomic data as set forth in claim 25, further including means for generating a new partition scheme with one partition representing the number of data samples in the united group and the other partition representing the number of data samples in the set of proteomic and genomic data minus the number of data samples in the united group.

27. An apparatus for plotting proteomic and genomic data as set forth in claim 23, further comprising means for:
a. receiving a user selection of multiple partition charts;
b. receiving a user request to intersect the partition charts into an intersected group consisting of data present in all of the multiple partition charts selected by the user; and
c. generating a list of the data in the intersected group.

28. An apparatus for plotting proteomic and genomic data as set forth in claim 27, further including means for generating and displaying a chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single chart.

29. An apparatus for plotting proteomic and genomic data as set forth in claim 28, further including means for generating a new partition scheme with one partition representing the number of data samples in the intersected group and the other partition representing the number of data samples in the set of proteomic and genomic data minus the number of data samples in the intersected group.

30. An apparatus for plotting proteomic and genomic data as set forth in claim 23, further including means for displaying a list of data used for the generation of a particular partition chart when the partition chart is selected; whereby a user may view a partition chart and may also examine the underlying data.

31. An apparatus for plotting proteomic and genomic data as set forth in claim 30, further including means for receiving a user request to search for a particular chart of data and, in response to the user request, for indicating the charts in which the particular chart of data is present; whereby a user may visually determine which chart groups include the particular chart of data.

32. An apparatus for plotting proteomic and genomic data as set forth in claim 23, further comprising means for:
a. receiving a user selection of multiple partition charts;
b. receiving a user request to unite the partition charts into a united group consisting of the data present in all of the multiple partition charts selected by the user; and
c. generating a list of the data in the united group.

33. An apparatus for plotting proteomic and genomic data as set forth in claim 32, further including means for generating and displaying a chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single chart.

34. An apparatus for plotting proteomic and genomic data as set forth in claim 33, further including means for generating a new partition scheme with one partition representing the number of data samples in the united group and the other partition representing the number of data samples in the set of proteomic and genomic data minus the number of data samples in the united group.

35. An apparatus for plotting proteomic and genomic data as set forth in claim 34, further comprising means for:
a. receiving a user selection of multiple partition charts;
b. receiving a user request to intersect the partition charts into an intersected group consisting of data present in all of the multiple partition charts selected by the user; and
c. generating a list of the data in the intersected group.

36. An apparatus for plotting proteomic and genomic data as set forth in claim 35, further including means for generating and displaying a chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single chart.

37. An apparatus for plotting proteomic and genomic data as set forth in claim 36, further including means for generating a new partition scheme with one partition representing the number of data samples in the intersected group and the other partition representing the number of data samples in the set of proteomic and genomic data minus the number of data samples in the intersected group.

38. An apparatus for plotting proteomic and genomic data as set forth in claim 37, further including means for displaying a list of data used for the generation of a particular partition chart when the partition chart is selected; whereby a user may view a partition chart and may also examine the underlying data.

39. An apparatus for plotting proteomic and genomic data as set forth in claim 38, further including means for receiving a user request to search for a particular chart of data and, in response to the user request, for indicating the charts in which the particular chart of data is present; whereby a user may visually determine which chart groups include the particular chart of data.

40. A method for plotting proteomic and genomic data, the method operating on a computer system including a processor, a memory coupled with the processor, an input coupled with the processor for receiving proteomic and genomic data and for receiving user input, and an output coupled with the processor for displaying data in a visual form, the method comprising the steps of:
a. receiving a set of proteomic and genomic data in the input of the computer system including data samples with characteristics;
b. receiving at least one partitioning scheme in the input of the computer system, with each partition scheme including at least one partition into which a portion of the data is to be grouped based on a characteristic;
c. generating a graphical representation of the relative size of each partition in each partition scheme;
d. accepting a user selection of a particular partition scheme in the input of the computer system; and, in response,
e. adjusting the view of all other partition schemes to reflect the distribution of the characteristic used as the basis of the selected partition scheme; and
f. outputting a graphical representation through the output of the computer system for display in a visual display device; whereby a user can view a graphical representation of the distribution of a particular characteristic based on a particular partitioning scheme.

41. A method for plotting proteomic and genomic data as set forth in claim 40, wherein the step of generating a graphical representation generates a graphical representation in the form of pie-type charts, each with pie slices, with each pie slice representing the portion of the overall pie attributable to a particular partition of a partition scheme.

42. A method for plotting proteomic and genomic data as set forth in claim 40, wherein the step of generating a graphical representation generates a graphical representation in the form of a plurality of pie chart groups, with each pie chart group including a partition scheme pie chart representing the overall distribution a characteristic selected, and a plurality of partition pie charts, each representing a partition within the partition scheme pie chart.

43. A method for plotting proteomic and genomic data as set forth in claim 42, wherein the step of accepting a user selection of a particular partition scheme accepts a user selection of a particular partition scheme by the user graphically selecting a pie chart group among the plurality of pie chart groups, and wherein the step of adjusting the view of all other partition schemes to reflect the distribution of the characteristic used as the basis of the selected partition scheme adjusts all of the pie chart groups such that the partition scheme pie charts and the partition pie charts are all partitioned according to the partition scheme of the selected pie chart group; whereby the pie charts may be compared visually to determine possible correlations therebetween.

44. A method for plotting proteomic and genomic data as set forth in claim 43, further comprising means for:
a. receiving a user selection of multiple partition pie charts;
b. receiving a user request to unite the partition pie charts into a united group consisting of the data present in all of the multiple partition pie charts selected by the user; and
c. generating a list of the data in the united group.

45. A method for plotting proteomic and genomic data as set forth in claim 44, further including a step of generating and displaying a pie chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single pie chart.

46. A method for plotting proteomic and genomic data as set forth in claim 45, further including a step of generating a new partition scheme with one partition representing the number of data samples in the united group and the other partition representing the number of data samples in the set of proteomic and genomic data minus the number of data samples in the united group.

47. A method for plotting proteomic and genomic data as set forth in claim 43, further comprising steps of:
a. receiving a user selection of multiple partition pie charts;
b. receiving a user request to intersect the partition pie charts into an intersected group consisting of data present in all of the multiple partition pie charts selected by the user; and
c. generating a list of the data in the intersected group.

48. A method for plotting proteomic and genomic data as set forth in claim 47, further including steps of generating and displaying a pie chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single pie chart.

49. A method for plotting proteomic and genomic data as set forth in claim 48, further including steps of generating a new partition scheme with one partition representing the number of data samples in the intersected group and the other partition representing the number of data samples in the set of proteomic and genomic data minus the number of data samples in the intersected group.

50. A method for plotting proteomic and genomic data as set forth in claim 43, further including a step of displaying a list of data used for the generation of a particular partition pie chart when the partition pie chart is selected; whereby a user may view a partition pie chart and may also examine the underlying data.

51. A method for plotting proteomic and genomic data as set forth in claim 40, further including a steps of receiving a user request to search for a particular piece of data and, in response to the user request, of indicating the pie charts in which the particular piece of data is present; whereby a user may visually determine which pie chart groups include the particular piece of data.

52. A method for plotting proteomic and genomic data as set forth in claim 43, further comprising steps of:
a. receiving a user selection of multiple partition pie charts;
b. receiving a user request to unite the partition pie charts into a united group consisting of the data present in all of the multiple partition pie charts selected by the user; and
c. generating a list of the data in the united group.

53. A method for plotting proteomic and genomic data as set forth in claim 53, further including steps of generating and displaying a pie chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single pie chart.

54. A method for plotting proteomic and genomic data as set forth in claim 53, further including a step of generating a new partition scheme with one partition representing the number of data samples in the united group and the other partition representing the number of data samples in the set of proteomic and genomic data minus the number of data samples in the united group.

55. A method for plotting proteomic and genomic data as set forth in claim 54, further comprising steps of:
a. receiving a user selection of multiple partition pie charts;
b. receiving a user request to intersect the partition pie charts into an intersected group consisting of data present in all of the multiple partition pie charts selected by the user; and
c. generating a list of the data in the intersected group.

56. A method for plotting proteomic and genomic data as set forth in claim 55, further including steps of generating and displaying a pie chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single pie chart.

57. A method for plotting proteomic and genomic data as set forth in claim 56, further including a step of generating a new partition scheme with one partition representing the number of data samples in the intersected group and the other partition representing the number of data samples in the set of proteomic and genomic data minus the number of data samples in the intersected group.

58. A method for plotting proteomic and genomic data as set forth in claim 57, further including a step of displaying a list of data used for the generation of a particular partition pie chart when the partition pie chart is selected; whereby a user may view a partition pie chart and may also examine the underlying data.

59. A method for plotting proteomic and genomic data as set forth in claim 58, further including steps of receiving a user request to search for a particular piece of data and, in response to the user request, of indicating the pie charts in which the particular piece of data is present; whereby a user may visually determine which pie chart groups include the particular piece of data.

60. A method for plotting proteomic and genomic data as set forth in claim 40, wherein step of generating a graphical representation generates a graphical representation in the form of a graphical chart, each with chart slices, with each chart slice representing the portion of the overall chart attributable to a particular partition of a partition scheme.

61. A method for plotting proteomic and genomic data as set forth in claim 40, wherein the step of generating a graphical representation generates a graphical representation in the form of a plurality of chart groups, with each chart group including a partition scheme chart representing the overall distribution a characteristic selected, and a plurality of partition charts, each representing a partition within the partition scheme chart.

62. A method for plotting proteomic and genomic data as set forth in claim 61, wherein the step of accepting a user selection of a particular partition scheme accepts a user selection of a particular partition scheme by the user graphically selecting a chart group among the plurality of chart groups, and wherein the step of adjusting the view of all other partition schemes to reflect the distribution of the characteristic used as the basis of the selected partition scheme adjusts all of the chart groups such that the partition scheme charts and the partition charts are all partitioned according to the partition scheme of the selected chart group; whereby the charts may be compared visually to determine possible correlations therebetween.

63. A method for plotting proteomic and genomic data as set forth in claim 62, further comprising steps of:
a. receiving a user selection of multiple partition charts;
b. receiving a user request to unite the partition charts into a united group consisting of the data present in all of the multiple partition charts selected by the user; and
c. generating a list of the data in the united group.

64. A method for plotting proteomic and genomic data as set forth in claim 63, further including a step of generating and displaying a chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single chart.

65. A method for plotting proteomic and genomic data as set forth in claim 64, further including a step of generating a new partition scheme with one partition representing the number of data samples in the united group and the other partition representing the number of data samples in the set of proteomic and genomic data minus the number of data samples in the united group.

66. A method for plotting proteomic and genomic data as set forth in claim 62, further comprising steps of:
a. receiving a user selection of multiple partition charts;
b. receiving a user request to intersect the partition charts into an intersected group consisting of data present in all of the multiple partition charts selected by the user; and
c. generating a list of the data in the intersected group.

67. A method for plotting proteomic and genomic data as set forth in claim 66, further including steps for generating and displaying a chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single chart.

68. A method for plotting proteomic and genomic data as set forth in claim 67, further including a step for generating a new partition scheme with one partition representing the number of data samples in the intersected group and the other partition representing the number of data samples in the set of proteomic and genomic data minus the number of data samples in the intersected group.

69. A method for plotting proteomic and genomic data as set forth in claim 62, further including a step of displaying a list of data used for the generation of a particular partition chart when the partition chart is selected; whereby a user may view a partition chart and may also examine the underlying data.

70. A method for plotting proteomic and genomic data as set forth in claim 69, further including steps of receiving a user request to search for a particular chart of data and, in response to the user request, of indicating the charts in which the particular chart of data is present; whereby a user may visually determine which chart groups include the particular chart of data.

71. A method for plotting proteomic and genomic data as set forth in claim 62, further comprising steps of:
a. receiving a user selection of multiple partition charts;
b. receiving a user request to unite the partition charts into a united group consisting of the data present in all of the multiple partition charts selected by the user; and
c. generating a list of the data in the united group.

72. A method for plotting proteomic and genomic data as set forth in claim 71, further including steps of generating and displaying a chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single chart.

73. A method for plotting proteomic and genomic data as set forth in claim 72, further including a step of generating a new partition scheme with one partition representing the number of data samples in the united group and the other partition representing the number of data samples in the set of proteomic and genomic data minus the number of data samples in the united group.

74. A method for plotting proteomic and genomic data as set forth in claim 73, further comprising steps of:
a. receiving a user selection of multiple partition charts;
b. receiving a user request to intersect the partition charts into an intersected group consisting of data present in all of the multiple partition charts selected by the user; and
c. generating a list of the data in the intersected group.

75. A method for plotting proteomic and genomic data as set forth in claim 74, further including steps for generating and displaying a chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single chart.

76. A method for plotting proteomic and genomic data as set forth in claim 75, further including a step of generating a new partition scheme with one partition representing the number of data samples in the intersected group and the other partition representing the number of data samples in the set of proteomic and genomic data minus the number of data samples in the intersected group.

77. A method for plotting proteomic and genomic data as set forth in claim 76, further including a step of displaying a list of data used for the generation of a particular partition chart when the partition chart is selected; whereby a user may view a partition chart and may also examine the underlying data.

78. A method for plotting proteomic and genomic data as set forth in claim 77, further including steps of receiving a user request to search for a particular chart of data and, in response to the user request, of indicating the charts in which the particular chart of data is present; whereby a user may visually determine which chart groups include the particular chart of data.

79. A computer program product for plotting proteomic and genomic data, a computer system including a processor, a memory coupled with the processor, an input coupled with the processor for receiving proteomic and genomic data and for receiving user input, and an output coupled with the processor for displaying data in a visual form the computer program product comprising means, stored on a computer readable medium, for:
a. receiving a set of proteomic and genomic data including data samples with characteristics;
b. receiving at least one partitioning scheme, with each partition scheme including at least one partition into which a portion of the data is to be grouped based on a characteristic;
c. generating a graphical representation of the relative size of each partition in each partition scheme;
d. accepting a user selection of a particular partition scheme; and, in response,
e. adjusting the view of all other partition schemes to reflect the distribution of the characteristic used as the basis of the selected partition scheme; and
f. outputting a graphical representation; whereby a user can view a graphical representation of the distribution of a particular characteristic based on a particular partitioning scheme.

80. A computer program product for plotting proteomic and genomic data as set forth in claim 79, wherein the means for generating a graphical representation generates a graphical representation in the form of pie-type charts, each with pie slices, with each pie slice representing the portion of the overall pie attributable to a particular partition of a partition scheme.

81. A computer program product for plotting proteomic and genomic data as set forth in claim 79, wherein the means for generating a graphical representation generates a graphical representation in the form of a plurality of pie chart groups, with each pie chart group including a partition scheme pie chart representing the overall distribution a characteristic selected, and a plurality of partition pie charts, each representing a partition within the partition scheme pie chart.

82. A computer program product for plotting proteomic and genomic data as set forth in claim 81, wherein the means for accepting a user selection of a particular partition scheme accepts a user selection of a particular partition scheme by the user graphically selecting a pie chart group among the plurality of pie chart groups, and wherein the means for adjusting the view of all other partition schemes to reflect the distribution of the characteristic used as the basis of the selected partition scheme adjusts all of the pie chart groups such that the partition scheme pie charts and the partition pie charts are all partitioned according to the partition scheme of the selected pie chart group; whereby the pie charts may be compared visually to determine possible correlations therebetween.

83. A computer program product for plotting proteomic and genomic data as set forth in claim 82, further comprising means for:
a. receiving a user selection of multiple partition pie charts;
b. receiving a user request to unite the partition pie charts into a united group consisting of the data present in all of the multiple partition pie charts selected by the user; and
c. generating a list of the data in the united group.

84. A computer program product for plotting proteomic and genomic data as set forth in claim 83, further including means for generating and displaying a pie chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single pie chart.

85. A computer program product for plotting proteomic and genomic data as set forth in claim 84, further including means for generating a new partition scheme with one partition representing the number of data samples in the united group and the other partition representing the number of data samples in the set of proteomic and genomic data minus the number of data samples in the united group.

86. A computer program product for plotting proteomic and genomic data as set forth in claim 82, further comprising means for:
a. receiving a user selection of multiple partition pie charts;
b. receiving a user request to intersect the partition pie charts into an intersected group consisting of data present in all of the multiple partition pie charts selected by the user; and
c. generating a list of the data in the intersected group.

87. A computer program product for plotting proteomic and genomic data as set forth in claim 86, further including means for generating and displaying a pie chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single pie chart.

88. A computer program product for plotting proteomic and genomic data as set forth in claim 87, further including means for generating a new partition scheme with one partition representing the number of data samples in the intersected group and the other partition representing the number of data samples in the set of proteomic and genomic data minus the number of data samples in the intersected group.

89. A computer program product for plotting proteomic and genomic data as set forth in claim 82, further including means for displaying a list of data used for the generation of a particular partition pie chart when the partition pie chart is selected; whereby a user may view a partition pie chart and may also examine the underlying data.

90. A computer program product for plotting proteomic and genomic data as set forth in claim 79, further including means for receiving a user request to search for a particular piece of data and, in response to the user request, for indicating the pie charts in which the particular piece of data is present; whereby a user may visually determine which pie chart groups include the particular piece of data.

91. A computer program product for plotting proteomic and genomic data as set forth in claim 82, further comprising means for:
a. receiving a user selection of multiple partition pie charts;
b. receiving a user request to unite the partition pie charts into a united group consisting of the data present in all of the multiple partition pie charts selected by the user; and
c. generating a list of the data in the united group.

92. A computer program product for plotting proteomic and genomic data as set forth in claim 91, further including means for generating and displaying a pie chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single pie chart.

93. A computer program product for plotting proteomic and genomic data as set forth in claim 92, further including means for generating a new partition scheme with one partition representing the number of data samples in the united group and the other partition representing the number of data samples in the set of proteomic and genomic data minus the number of data samples in the united group.

94. A computer program product for plotting proteomic and genomic data as set forth in claim 93, further comprising means for:
a. receiving a user selection of multiple partition pie charts;
b. receiving a user request to intersect the partition pie charts into an intersected group consisting of data present in all of the multiple partition pie charts selected by the user; and
c. generating a list of the data in the intersected group.

95. A computer program product for plotting proteomic and genomic data as set forth in claim 94, further including means for generating and displaying a pie chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single pie chart.

96. A computer program product for plotting proteomic and genomic data as set forth in claim 95, further including means for generating a new partition scheme with one partition representing the number of data samples in the intersected group and the other partition representing the number of data samples in the set of proteomic and genomic data minus the number of data samples in the intersected group.

97. A computer program product for plotting proteomic and genomic data as set forth in claim 96, further including means for displaying a list of data used for the generation of a particular partition pie chart when the partition pie chart is selected; whereby a user may view a partition pie chart and may also examine the underlying data.

98. A computer program product for plotting proteomic and genomic data as set forth in claim 97, further including means for receiving a user request to search for a particular piece of data and, in response to the user request, for indicating the pie charts in which the particular piece of data is present; whereby a user may visually determine which pie chart groups include the particular piece of data.

99. A computer program product for plotting proteomic and genomic data as set forth in claim 79, wherein the means for generating a graphical representation generates a graphical representation in the form of a graphical chart, each with chart slices, with each chart slice representing the portion of the overall chart attributable to a particular partition of a partition scheme.

100. A computer program product for plotting proteomic and genomic data as set forth in claim 79, wherein the means for generating a graphical representation generates a graphical representation in the form of a plurality of chart groups, with each chart group including a partition scheme chart representing the overall distribution a characteristic selected, and a plurality of partition charts, each representing a partition within the partition scheme chart.

101. A computer program product for plotting proteomic and genomic data as set forth in claim 100, wherein the means for accepting a user selection of a particular partition scheme accepts a user selection of a particular partition scheme by the user graphically selecting a chart group among the plurality of chart groups, and wherein the means for adjusting the view of all other partition schemes to reflect the distribution of the characteristic used as the basis of the selected partition scheme adjusts all of the chart groups such that the partition scheme charts and the partition charts are all partitioned according to the partition scheme of the selected chart group; whereby the charts may be compared visually to determine possible correlations therebetween.

102. A computer program product for plotting proteomic and genomic data as set forth in claim 101, further comprising means for:
a. receiving a user selection of multiple partition charts;
b. receiving a user request to unite the partition charts into a united group consisting of the data present in all of the multiple partition charts selected by the user; and
c. generating a list of the data in the united group.

103. A computer program product for plotting proteomic and genomic data as set forth in claim 102, further including means for generating and displaying a chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single chart.

104. A computer program product for plotting proteomic and genomic data as set forth in claim 103, further including means for generating a new partition scheme with one partition representing the number of data samples in the united group and the other partition representing the number of data samples in the set of proteomic and genomic data minus the number of data samples in the united group.

105. A computer program product for plotting proteomic and genomic data as set forth in claim 104, further comprising means for:
a. receiving a user selection of multiple partition charts;
b. receiving a user request to intersect the partition charts into an intersected group consisting of data present in all of the multiple partition charts selected by the user; and
c. generating a list of the data in the intersected group.

106. A computer program product for plotting proteomic and genomic data as set forth in claim 105, further including means for generating and displaying a chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single chart.

107. A computer program product for plotting proteomic and genomic data as set forth in claim 106, further including means for generating a new partition scheme with one partition representing the number of data samples in the intersected group and the other partition representing the number of data samples in the set of proteomic and genomic data minus the number of data samples in the intersected group.

108. A computer program product for plotting proteomic and genomic data as set forth in claim 101, further including means for displaying a list of data used for the generation of a particular partition chart when the partition chart is selected; whereby a user may view a partition chart and may also examine the underlying data.

109. A computer program product for plotting proteomic and genomic data as set forth in claim 108, further including means for receiving a user request to search for a particular chart of data and, in response to the user request, for indicating the charts in which the particular chart of data is present; whereby a user may visually determine which chart groups include the particular chart of data.

110. A computer program product for plotting proteomic and genomic data as set forth in claim 101, further comprising means for:
a. receiving a user selection of multiple partition charts;
b. receiving a user request to unite the partition charts into a united group consisting of the data present in all of the multiple partition charts selected by the user; and
c. generating a list of the data in the united group.

111. A computer program product for plotting proteomic and genomic data as set forth in claim 110, further including means for generating and displaying a chart based on the data in the united group and partitioned into two slices, with one slice representing the number of data samples in the united group and the other slice representing the number of data samples in the set of proteomic and genomic data minus the number of data samples in the united group; whereby a user may view the number of samples in the united group and its complement in a single chart.

112. A computer program product for plotting proteomic and genomic data as set forth in claim 111, further including means for generating a new partition scheme with one partition representing the number of data samples in the united group and the other partition representing the number of data samples in the set of proteomic and genomic data minus the number of data samples in the united group.

113. A computer program product for plotting proteomic and genomic data as set forth in claim 112, further comprising means for:
a. receiving a user selection of multiple partition charts;
b. receiving a user request to intersect the partition charts into an intersected group consisting of data present in all of the multiple partition charts selected by the user; and
c. generating a list of the data in the intersected group.

114. A computer program product for plotting proteomic and genomic data as set forth in claim 113, further including means for generating and displaying a chart based on the data in the intersected group and partitioned into two slices, with one slice representing the number of data samples in the intersected group and the other slice representing the number of data samples in the set of proteomic and genomic data minus the number of data samples in the intersected group; whereby a user may view the number of samples in the intersected group and its complement in a single chart.

115. A computer program product for plotting proteomic and genomic data as set forth in claim 114, further including means for generating a new partition scheme with one partition representing the number of data samples in the intersected group and the other partition representing the number of data samples in the set of proteomic and genomic data minus the number of data samples in the intersected group.

116. A computer program product for plotting proteomic and genomic data as set forth in claim 115, further including means for displaying a list of data used for the generation of a particular partition chart when the partition chart is selected; whereby a user may view a partition chart and may also examine the underlying data.

117. A computer program product for plotting proteomic and genomic data as set forth in claim 116, further including means for receiving a user request to search for a particular chart of data and, in response to the user request, for indicating the charts in which the particular chart of data is present; whereby a user may visually determine which chart groups include the particular chart of data.

118. An apparatus for plotting proteomic and genomic data, the apparatus comprising a computer system including a processor, a memory coupled with the processor, an input coupled with the processor for receiving proteomic and genomic data and for receiving user input, and an output coupled with the processor for displaying data in a visual form, wherein the computer system further comprises means, residing in its processor and memory, for:
a. receiving a set of proteomic and genomic data including data samples with characteristics;
b. receiving at least one partitioning scheme, with each partition scheme including at least one partition into which a portion of the data is to be grouped based on a characteristic;
c. generating a graphical representation of the relative size of each partition in each partition scheme; and
d. outputting a graphical representation; whereby a user can view a graphical representation of the distribution of a particular characteristic based on a particular partitioning scheme.

119. A method for plotting proteomic and genomic data, the method operating on a computer system including a processor, a memory coupled with the processor, an input coupled with the processor for receiving proteomic and genomic data and for receiving user input, and an output coupled with the processor for displaying data in a visual form, the method comprising the steps of:
a. receiving a set of proteomic and genomic data in the input of the computer system including data samples with characteristics;
b. receiving at least one partitioning scheme in the input of the computer system, with each partition scheme including at least one partition into which a portion of the data is to be grouped based on a characteristic;
c. generating a graphical representation of the relative size of each partition in each partition scheme; and
d. outputting a graphical representation through the output of the computer system for display in a visual display device; whereby a user can view a graphical representation of the distribution of a particular characteristic based on a particular partitioning scheme.

120. A computer program product for plotting proteomic and genomic data, a computer system including a processor, a memory coupled with the processor, an input coupled with the processor for receiving proteomic and genomic data and for receiving user input, and an output coupled with the processor for displaying data in a visual form the computer program product comprising means, stored on a computer readable medium, for:
a. receiving a set of proteomic and genomic data including data samples with characteristics;
b. receiving at least one partitioning scheme, with each partition scheme including at least one partition into which a portion of the data is to be grouped based on a characteristic;
c. generating a graphical representation of the relative size of each partition in each partition scheme; and
d. outputting a graphical representation; whereby a user can view a graphical representation of the distribution of a particular characteristic based on a particular partitioning scheme.
